(19) United States
(12) Patent Application Publication
(10) Pub. No.: US 2025/0014674 A1
(43) Pub. Date: Jan. 9, 2025

(54) METHOD FOR CYTOMETRIC ANALYSIS

(71) Applicants: METAFORA BIOSYSTEMS, Paris (FR); INRIA - INSTITUT NATIONAL DE RECHERCHE EN INFORMATIQUE ET EN AUTOMATIQUE, Le Chesnay (FR); UNIVERSITE PARIS-SACLAY, Gif-sur-Yvette (FR)

(72) Inventors: Marc GLISSE, Sceaux (FR); Louis PUJOL, Bures-sur-Yvette (FR); Baptiste LABARTHE, Paris (FR); Romain TAILHADES, Bordeaux (FR); Kamila CZECHOWSKA-KUSIO, Rothenthurm (CH); Dominique BLANCHARD, Meudon (FR); Vincent PETIT, Paris (FR); Mélanie RENNOU, Gometz-le-Châtel (FR)

(73) Assignees: METAFORA BIOSYSTEMS, Paris (FR); INRIA - INSTITUT NATIONAL DE RECHERCHE EN INFORMATIQUE ET EN AUTOMATIQUE, Le Chesnay (FR); UNIVERSITE PARIS-SACLAY, Gif-sur-Yvette (FR)

(21) Appl. No.: 18/713,362

(22) PCT Filed: Nov. 25, 2022
(86) PCT No.: PCT/EP2022/083349

§ 371 Date: May 24, 2024

(30) Foreign Application Priority Data
Nov. 25, 2021 (EP) 21306640.0

(51) Int. Cl. G16B 15/00; G16B 45/00

(52) U.S. Cl. CPC G16B 15/00; G16B 45/00

(57) ABSTRACT

A device and a computer-implemented method for analyzing a dataset associated with a plurality of biological objects selected from cells, cell-derived vesicles, acellular microorganisms, and/or biofunctionalized materials; the dataset including N cytometric events, each associated with a biological object, each cytometric event being defined by at least two cytometric parameters measured for the corresponding biological object so that the dataset is represented by a cloud of N points in a D-dimensional space; the device and method being configured to output at least the hierarchical structure representing the different classes of biological objects and their mutual relationships.

### FIELD OF THE INVENTION

The present invention relates to the general field of cytometric data analysis.

### BACKGROUND

The emergence of protein sequencing, followed by DNA sequencing, and the development of automatic sequencers, have revolutionized biology. The classic descriptive and reductionist approach (one gene, one messenger RNA, one protein) has been succeeded by a more comprehensive understanding of biological systems based on the analysis of sets of biological elements (“-omes”). The basic idea associated with “omics” approaches is to grasp the complexity of living organisms as a whole, using methodologies that are as unrestricted as possible in their descriptive scope.

Such approaches mainly comprise: genomics (the study of genes), transcriptomics (analysis of gene expression and its regulation), proteomics (study of proteins), metabolomics (analysis of metabolites), and metabonomics (study of in vivo metabolic profiles).

Genomics is divided into two branches: structural genomics, which focuses on whole genome sequencing, and functional genomics, which aims to determine the function and expression of sequenced genes. In functional genomics, techniques are applied to a large number of genes in parallel: for example, the phenotype of mutants can be analyzed for an entire gene family, or the expression of all genes in an entire organism.

Transcriptomics is the study of all messenger RNAs produced during the transcription process of a genome. It relies on the quantification of all these messenger RNAs, providing a relative indication of the transcription rate of different genes under given conditions.

Proteomics is the analysis of all the proteins in an organelle, cell, tissue, organ, or organism under given conditions. Proteomics aims to globally identify proteins extracted from a cell culture, tissue, or biological fluid, their localization in cellular compartments, their possible post-translational modifications, as well as their quantity. It allows quantifying variations in their expression levels, for example, depending on time, environment, developmental stage, physiological and pathological state, species of origin, etc. It also studies the interactions that proteins have with other proteins, with DNA or RNA, or other substances.

Metabolomics studies all metabolites (sugars, amino acids, fatty acids, etc.), such as metabolic substrates, intermediates, products, as well as hormones, other signaling molecules, and secondary metabolites, present in a cell, organ, or organism.

Metabonomics involves monitoring in vivo metabolic profiles, providing information on the toxicity of drugs, pathological processes, and gene function.

Cytomics corresponds to the analysis of the cytome, the ensemble of cellular constituents, including morphological, antigenic, and functional characteristics, which allow defining or modeling the state and functioning of a cell at a given time. Cytome analysis allows describing the structural and functional heterogeneity of the various cells in an organism.

The aforementioned approaches provide a wealth of information on cellular and/or tissue response to in vitro or in vivo exposure. They can be particularly useful in identifying and characterizing new biomarkers (for diagnosis, susceptibility, prognosis, exposure, effect), generating new mechanistic knowledge (modes of action), or developing new tools for predictive efficacy or toxicology to aid in the identification of new therapeutic targets or candidate drugs or vaccines.

The automation of sequencing techniques and the development of high-throughput techniques, made possible notably by the emergence of specialized technological platforms, have enabled the industrialization of data production and the simultaneous analysis of a large number of variables.

This results in a vast amount of data to be processed, analyzed, visualized, and interpreted in the most informative manner possible to extract the maximum information about the biological process or system under study.

From a biostatistical perspective, data obtained through “omics” approaches concern a large number of variables that need to be analyzed jointly. For example, transcriptomic analyses allow studying the expression of several thousand genes simultaneously.

It is therefore desirable to have powerful biostatistical and bioinformatics tools to process, analyze, and interpret the mass of data generated by “omics” approaches.

There are numerous techniques for acquiring “omics” data, such as, for example, mass spectrometry techniques, chromatography, sequencing, spectral cytometry, mass cytometry, flow cytometry, image cytometry . . . .

Cytometry refers to a set of techniques for analyzing a biological sample comprising a set of “biological elements,” such as cells, vesicles, or particles, in suspension.

Cytometry techniques are essential analytical techniques for the identification and characterization of a population of cells, vesicles, or particles. Moreover, cytomics can address the question of intracellular or extracellular processes and intercellular interactions because cytomics provides a single-cell view of more complex cellular conglomerates, such as tissues in multicellular organisms or communities in the case of unicellular bacteria, yeast, algae, etc.

The reliability and reproducibility of results are paramount; however, a major source of variation in cytometry lies in data analysis.

Conventional cytometry data analysis typically involves sequential manual selection (or “gating”) of regions of interest, usually in two-dimensional scatterplots or contour plots, representing one parameter on each axis. This is the case, for example, when analyzing flow cytometry data. Analysis is straightforward with three or four-color immunofluorescence data, but becomes much more complex when examining an increasing number of cellular markers, leading to increased variability related to human operator and reproducibility issues.

Current state-of-the-art cytometry techniques are capable of measuring dozens, hundreds, or even thousands of parameters, thus generating complex multidimensional datasets whose manual analysis is time-consuming. Manual segmentation is therefore subjective, laborious, unreliable, and costly.

Segmentation can be automated, with segments defined using software, without operator supervision. It can involve artificial intelligence, but the criteria for grouping cytometric events are then no longer understandable by the operator. Alternatively, it can rely on analysis algorithms.

Several comparative studies have evaluated unsupervised clustering methods based on their ability to reproduce manual selection (or segmentation or “gating”) and to detect rare cell populations, as well as their execution time. Although some methods may be effective for certain uses, none meet all the requirements to be used with confidence routinely by users.

Furthermore, automated techniques allow cytometric analysis without manual variability, subjectivity, and segmentation bias, and therefore new methods have been developed in this field over the last decade. However, many automated techniques still require parameterization of algorithms requiring operator intervention, introducing a significant amount of human subjectivity and thus result non-reproducibility.

As a result, the adoption of automated analysis among researchers in universities, biotechnology, the pharmaceutical industry, or clinical research has been slow, and manual segmentation remains the default method and the norm.

The main reasons why clinical centers do not use automated analysis have recently been identified as a lack of confidence or understanding of the algorithms used and a lack of resources. For automated analysis techniques to replace manual segmentation, not only must the identification results of cell populations correspond to those of an expert manual analysis, but the results obtained from the algorithms must also be robust, meaning perfectly reproducible.

In the specific example of flow cytometry, the biological fluid containing the cells, vesicles, or particles to be analyzed flows continuously past a detector. For each cell, vesicle, or particle passing in front of the detector, it is possible to measure values for cytometric parameters.

In general, a “cytometric event” is a vector associated with a particle that gathers the measured values of cytometric parameters for that particle in an ordered manner.

Conventionally, a cytometric event gathers, for each particle, values for more than 10 cytometric parameters. Each value of a cytometric parameter, for a particle, is called a “cytometric measurement.”

To analyze all cytometric measurements, points representing each cytometric event can be presented in a space of one, two, or three dimensions on “presentation graphs.” Each dimension corresponds to a cytometric parameter. Graphs representing cytometric events are thus “projections” that allow visualizing one, two, or three cytometric measurements for each cytometric event.

To facilitate analysis, it is necessary to “segment” point clouds, i.e., to create groups or “segments” bringing together cytometric events with similar values for several cytometric parameters. The analysis of the segments thus obtained, for example, the analysis of their respective weights, allows characterizing the biological fluid, for example, to detect a biological anomaly or a pathological risk.

When segmentation is done manually, the operator defines the outlines of the segments on the presentation graphs.

Replacing manual segmentation with unsupervised automatic segmentation remains a major issue in the field of cytometry. The following four criteria are generally studied to assess the quality of an automatic segmentation method:

a) Correlation with Manual Segmentation:

The first criterion is a performance criterion, i.e., strong correlation with manual segmentation resulting from expert consensus on a well-known dataset. An expert should be able to easily find the main populations of interest in an automatic segmentation performed by an algorithm.

### b) Simultaneous Consideration of all Dimensions:

The second criterion concerns the simultaneous consideration of all dimensions of the data, or a set of dimensions among them. Indeed, in manual segmentation, the segments are made on the data projected onto pairs of markers, at best on triplets. By limiting oneself to this type of representation, part of the richness and complexity of the data and the structures that compose it are not taken into account. Considering multiple or all dimensions at the same time allows considering all the complexity of the data and, for example, discovering populations not identified by manual segmentation.

### c) Segmentation Hierarchy:

When the expert manually segments a cytometry file, he proceeds recursively by identifying increasingly granular populations. This hierarchical approach is particularly interesting because Biology itself classifies cells according to a hierarchical approach. Hierarchical segmentation allows adapting the level of granularity according to the expert's needs.

### d) Explainability and Robustness of the Algorithm:

The explainability of algorithms is a major point in artificial intelligence, and even more so in medical or research applications, where the application needs strong theoretical guarantees. Moreover, the robustness and repeatability of results on two identical datasets are also major challenges in medical and diagnostic applications.

Many methods exist to automatically and unsupervisedly segment cytometry data. However, none of them completely meet the four criteria outlined above, and none have been implemented in solutions allowed to be marketed in regulated markets.

Regarding correlation with manual segmentation (a), most methods have some level of correlation with expert segmentation, however, few achieve levels of performance and consistency stable according to the existing variety in datasets. Methods providing the best results are generally not compatible with explainability requirements.

Concerning the simultaneous consideration of all dimensions (b), most algorithms use all dimensions simultaneously. However, some methods do not, but for example, simply project the data onto predefined pairs of markers. These methods have the advantage of closely matching what the expert does but provide almost no added value in discovering more relevant populations.

Regarding hierarchical segmentation (c), few algorithms in the state of the art naturally generate hierarchical segmentation. Indeed, in general in statistical learning, segmentation methods (clustering) are mostly not hierarchical. This bias directly affects cytometry segmentation methods derived from statistical learning.

To overcome this problem, hierarchical segmentation is generally generated after the initial segmentation. The approach consists of asking the algorithm for a large number of clusters (i.e., groupings) at first and then applying a hierarchical segmentation method directly on the clusters and no longer on the points. This approach does generate hierarchical segmentation but has the disadvantage of decoupling the procedure into two distinct times which tends to create an artifact effect. Moreover, the stability of the hierarchical structure depending on the number of groups initially requested by the method is problematic.

Finally, regarding the explainability and robustness of algorithms (d), again few methods meet the criterion. On the one hand, many algorithms depend on random initialization and/or parameters. It is therefore usually necessary to rerun these algorithms several times to ensure results. On the other hand, the explainability of algorithms remains a major issue and generally, the most explainable algorithms are the simplest and often the least performing.

Furthermore, regardless of the segmentation method, the segments resulting from the analysis of a projection must be comparable with those resulting from the analysis of other projections. For example, after isolating in a first projection, cytometric events presenting values in predefined ranges for first and second cytometric parameters, and thus creating a segment of interest, it may be useful to represent specifically the cytometric events of this segment of interest, in a second projection.

The efficiency of the analysis is intimately linked to the operator's ability to make such comparisons.

Consequently, there is a need for a method for analyzing cytometric measurements implementing automated segmentation, which provides reliable and reproducible results, which is easy to implement and robust, and whose operation is understandable to humans (which may allow human intervention or verification by the user if necessary).

The invention aims to address, at least in part, this need.

The invention also allows the user to obtain a level of segmentation precision “on demand” in certain areas of the data structure.

### SUMMARY

Therefore, the invention relates to a computer-implemented method for analyzing a set of data associated with a plurality of biological objects chosen from cells, cellular origin vesicles, acellular microorganisms, and/or biofunctionalized materials; said set of data comprising N cytometric events, each associated with a biological object, each cytometric event being defined by at least two cytometric parameters measured for the corresponding biological object so that the set of data is represented by a cloud (X) of N points in a D-dimensional space.

The said method comprises:
determining, for each point (xᵢ) of said cloud (X), a density (w(xᵢ)) inversely proportional to the sum of distances, raised to a power D, between said point (xᵢ) and a set of neighboring points (Vₚ(xᵢ)) consisting of p points of said cloud (X) closest to said considered point (xᵢ),
segmenting said cloud of points (X) into modal segments (Mⱼ), each modal segment (Mⱼ) comprising a modal point (mⱼ), locally presenting a maximum density, and the points of the cloud belonging to the attraction basin of said modal point (mⱼ); the points of the attraction basin being recursively identified based on their density (w(xᵢ)) vis-à-vis the densities of their q nearest neighboring points;
for each modal segment (Mⱼ), determining a persistence (β), said persistence (β) being:
   of infinite value if no adjacent modal segment (Mₜ) to the modal segment (Mⱼ) has a point with a density greater than the density of its modal point (mⱼ); two or more modal segments being adjacent if they share a same density saddle greater than zero; otherwise
   representative of the depth of a density saddle between the modal segment (Mⱼ) and said at least one adjacent modal segment (Mₜ);
starting from a modal segment (M_{d}):
   determining which, among the considered one (Mⱼ) and its adjacent modal segment(s) (Mₜ), corresponds to the highest attraction basin so as to identify a hierarchical link between the considered modal segment (Mⱼ) and its adjacent modal segment(s) (Mₜ);
   fusing the considered modal segment (Mⱼ) and the modal segment(s) with which the considered modal segment (Mⱼ) has a persistence lower than or equal to a predefined persistence threshold, so as to define a parent modal segment having a higher attraction basin and having as modal point (mⱼ) the point presenting the maximum density among the points of the fused modal segments;
   iteratively repeating operations a) and b) starting at each new iteration from the parent modal segment;
   so as to determine a hierarchical structure defined based on persistence, said hierarchical structure comprising multiple levels (Lₖ), each level (Lₖ) defining a segmentation of the cloud (X) into a plurality of classes (Cₖⱼ), each class comprising all the points of one or more modal segments (Mⱼ) and being representative of a group of the plurality of biological objects;
outputting at least the hierarchical structure representing the different classes of biological objects and their mutual relationships.

As will be seen in more detail in the following description, the method advantageously employs a deterministic approach. Indeed, the method utilizes a density calculation based on distances between points of the cloud representing the entire dataset under study. The density calculated for a given point of the cloud is thus representative of a concentration of neighboring points around said point. The method described here thus differs from other methods using random metrics. For identical or nearly identical point clouds, substantially identical results are therefore obtained. Thus, the reproducibility and rigor of the results are ensured.

Furthermore, unlike artificial intelligence models, also known as “black boxes,” and other methods with steps of random nature (such as a random point selection step) or using random metrics, the algorithm implemented in the method of the invention is based on the analysis of cytometric data density, which enables explainability and traceability of the results proposed (i.e., modal segments in the hierarchical structure). This makes it particularly suitable for clinical analyses in which the principle of transparency is a fundamental prerequisite.

Finally, trials have shown that the segmentation criteria of the proposed method are particularly relevant for cytometric analysis.

Moreover, the method of the invention allows for the consideration of all dimensions present in the data (i.e., a set of cytometric data associated with a plurality of biological objects) to perform segmentation. Advantageously, the hierarchical structure provided as output by this method enables the expression of extreme variability in a biological sample.

Furthermore, the steps of the method are designed to replicate the hierarchical and multi-scale aspect of the data using topological analysis, which advantageously allows for obtaining a detailed representation of the native hierarchical structure of the cytometry-derived data, which is a different representation, for example, from a simple neighborhood graph. Such a neighborhood graph aims to replicate the overall topology of a point cloud and incorporates a notion of proximity between clusters, but does not incorporate a notion of hierarchy.

More specifically, the method described here exploits the calculated density values to reproduce a hierarchical and multi-scale aspect. Thus, for a hierarchical structure with multiple levels ranked from highest to lowest, high-density points will, for example, be represented at higher levels, while low-density points will be represented at lower levels in the hierarchical structure. The density value of a point is used and leveraged to position the point in the hierarchical structure, and possibly in a representation of this hierarchical structure. Thus, in the method described here, two points with very different densities will not be positioned at the same level. Typically, a rare cell, i.e., of low density, will be situated at a low level in the hierarchy. In other words, the hierarchical structure provided as output of the method translates the parental relationships between different levels consisting of classes. This allows for exploring these different levels as well as the classes constituting each level.

The method described here is not intended to ignore relative density differences between points in the cloud. On the contrary, the method exploits these relative differences to translate the hierarchical (i.e., parental) relationships between different levels. This method, therefore, distinguishes itself from, and is incompatible with, other methods aiming to reproduce a global topology of a point cloud and, for example, to identify clusters of points exhibiting similarity, but which at the same time erase the relative density differences between the points of the cloud, for example, through density-adaptive sampling. In the method described here, the density calculation performed is used to obtain the hierarchical structure, but not for sampling purposes.

As an illustration, if the space is in two dimensions, the points lie in a plane. If we represent density on a third dimension orthogonal to the plane, each modal segment corresponds to a mountain whose peak is the local maximum density associated with the modal point, and the valley between two adjacent mountains contains a density saddle. The saddle is the set of lowest points between two mountains belonging to the same ridge. The persistence of the mountain measures the difference in altitude between the bottom of the saddle with density greater than zero and the peak of this mountain. If a modal segment has low persistence, it can generally be represented by a bulge that protrudes slightly from the adjacent mountain, for example, on the flank of this adjacent mountain. The fusion step allows, for example, the fusion of such a bulge and this adjacent mountain. Consequently, the method of the invention advantageously avoids giving importance to disturbances in the density function caused by noise. Noise is associated with a low persistence value, and therefore, it will be fused with an adjacent modal segment for a reasonable choice of persistence threshold.

In one embodiment, the two parameters p and q are predetermined through a heuristic study on a large number of data. Thus, advantageously, the choice of these parameters does not rely on the operator, which ensures a method with analysis robustness independent of the operator.

The threshold persistence (i.e., persistence threshold) associated with a level determines the number of classes at that level, i.e., the granularity of the representation. The values of the threshold persistences allow choosing the numbers of classes associated with each level of the hierarchical structure. In other words, the choice of threshold persistence values allows simultaneously studying or analyzing different levels of the hierarchical structure, representing, for example, on the one hand, the general population formed by all the biological objects studied, and on the other hand, a subpopulation within the general population. The described method allows exploring the obtained hierarchical structure by defining different granularities for different areas of the hierarchical structure. In this aspect, the method described here differs from other methods where only one representation granularity, i.e., a uniform granularity, can be chosen. Thus, the method described here aims not only to determine a plurality of homogeneous main groupings but rather to have access to multi-scale data, i.e., data comprising subsets of data represented at different scales on the same representation. Access to multi-scale data, as in the method described here, allows accessing all the data at any time without loss of information. This allows the user to access at any time the data corresponding to a subpopulation of their choice, potentially more interesting and relevant to them.

In one embodiment, the point cloud segmentation step (X) into modal segments (Mⱼ) comprises, for each point (xᵢ), proceeding from the point with the highest density to the point with the lowest density:
comparing the density of said point (xᵢ) with that of its q nearest neighboring points (V_{q}(xᵢ)),
if the density of the considered point (x) is greater than the density of all its q nearest neighboring points (V_{q}(xᵢ)), defining a modal segment (Mⱼ) comprising the considered point (xᵢ), otherwise
including said considered point (xᵢ) in a modal segment comprising the point with the highest density among its q nearest neighboring points (V_{q}(xᵢ)).

In one embodiment, in the case of one or more adjacent modal segments (Mₜ) to the modal segment (Mⱼ), the persistence (β) is determined as a difference between the density of the modal point (mⱼ) and the highest density among the densities of the points in the density col between the modal segment (Mⱼ) and said at least one adjacent modal segment (Mₜ).

In one embodiment, the method comprises a preliminary step of receiving the set of data associated with a plurality of biological objects.

In one embodiment, the biological objects are:
animal, plant, fungal, protist, bacterial, or archaeobacterial origin cells,
cellular origin vesicles chosen from exosomes, ectosomes, microvesicles, microparticles, prostasomes, oncosomes, matrix/mineralization vesicles, or apoptotic bodies,
acellular microorganisms chosen from viruses, viroids, and prions, and/or
biofunctionalized materials comprising a synthetic or biological material chosen from a nanoparticle (such as a nanobead, nanosphere, or nanocapsule), a microparticle (such as a microbead, microsphere, or microcapsule), a lipid vesicle (such as a unilamellar vesicle, multilamellar vesicle, lipoplex, polyplex, lipopolyplex, liposome, niosome, cochleate, virosome, immunostimulating complex (ISCOM®)), said synthetic or biological material being coupled to, or coated with, one or more peptide(s), protein(s), antibody(ies), antibody fragment(s), receptor(s), cytokine(s), chemokine(s), toxin(s), oligonucleotide(s), colored or fluorescent molecule(s), amine, carboxyl, or hydroxyl group(s), bioactive molecule(s) (such as an immunomodulatory molecule, small molecule drug, peptidomimetic, medication), biotin, avidin, or streptavidin molecule(s), or a combination thereof.

In one embodiment, the cytometric parameters are selected from the size of the biological objects, their density, granularity, morphology, shape, refractive index, membrane composition, molecular content, content of a molecule, and/or level of expression of a molecule.

In one embodiment, one of the measured cytometric parameters is the level of expression of one or more protein(s), receptor(s), marker(s), and/or the level of expression of one or more nucleic acid(s) such as DNA or RNA.

In one embodiment, the cytometric data are obtained by flow cytometry (or FACS for “fluorescence activated cell sorting”), by PCR-activated cell sorting (or PACS), by microsphere affinity proteomics (MAP), by mass spectrometry, by chromatography, by CYTOF, by spectral cytometry, by mass cytometry, by imaging cytometry, by gene expression microarrays, by sequencing, for example of DNA (DNA-seq or single-cell (sc) DNA-seq) or RNA (RNA-seq or single-cell (sc) RNA-seq), by in situ hybridization, and/or by microscopy.

In one embodiment, the step of providing at least the hierarchical structure as output further comprises a display step. Indeed, as will be seen in more detail in the following description, the hierarchical structure obtained by the method offers particularly advantageous flexibility for visualizing and interpreting data from cytometric measurements.

In one embodiment, the display step of the hierarchical structure is configured to allow the operator to select one or more values of the predefined persistence threshold associated with at least one or more levels of the hierarchical structure. This allows the operator to choose with what degree of granularity they wish to display the cytometric data. The display step of the hierarchical structure can further be configured to allow the operator to directly select the classes to display, independently of the membership level.

In one embodiment, said display step comprises displaying:
at least one selection graph, said selection graph being a graphical representation of the classes and their respective relationships in the hierarchical structure, said selection graph being adapted to select at least one class by designating the corresponding class on said graphical representation;
at least one bi-dimensional or tri-dimensional presentation graph, each axis being associated with a respective cytometric parameter, said at least one presentation graph being configured to display the points of the cloud belonging to said at least one selected class using the selection graph.

Advantageously, this embodiment makes it particularly easy to navigate through the hierarchical structure, across all classes located at different levels. It allows for identifying relevant classes that were very difficult to identify with previous techniques. Moreover, the selection graph enables selecting classes at different levels to simultaneously visualize the points through the presentation graphs. In other words, the method of the invention makes it possible to visualize the “granularity” of the classes at one level but also to “zoom in” on one or more selected classes or to “zoom out” by selecting a class at a higher hierarchical level.

Furthermore, in one embodiment, the selection graph further comprises a visualization mean adapted to highlight said at least one selected class on the graphical representation.

Additionally, such simultaneous display, both of the hierarchical structure (i.e., selection graph) and the presentation graphs, advantageously presents to the operator the dataset in a format that includes global information about the membership of one or more groups of biological objects to one or more classes with close hierarchical links and specific information on the distribution of objects of the group(s) in relation to two or three cytometric parameters. This form of display not only has the simple advantage of conveying information in a way that an observer may intuitively find particularly appealing, clear, or logical. The simultaneous display of global (i.e., selection graph) and specific (i.e., presentation graphs) information provides the operator with contextual information enhancing the accuracy of data analysis. This combination of information at different scales gives the operator enriched information about cytometric data, including community relations among populations of biological objects. Not only does this display allow for easier and faster interpretation of the entire set of biological objects under analysis, but also, thanks to the additional information on the remaining links between subtypes of biological objects (i.e., selection graph) and the display of their cytological parameter values (i.e., presentation graphs), the operator can directly draw conclusions concerning, for example, clinical diagnosis, predicting the probability of a patient developing a disease, assessing a patient's response to treatment, or monitoring a patient under treatment and potentially adapting their treatment, etc. In general, especially in the case of cytometric data concerning a patient, the cognitive content of the information presented to the operator concerns the patient's health status. The operator can thus use this cognitive content to evaluate the patient's health status, make a diagnosis, propose treatment, or any other actions to improve the patient's health. In another example in which cytometric data concerns bio-functionalized materials, the operator can use the cognitive content of the invention's display during a process of designing or testing the effectiveness of candidate drugs in in vitro models.

Advantageously, it is possible to dynamically modify the graphical representation to navigate between different displays of the hierarchical structure, by the operator selecting the threshold values associated with at least one or more levels of the hierarchical structure, and by the operator selecting the classes to display. For example, at one moment, the operator can visualize a first subpopulation of biological objects. Then, at a later moment, the operator can visualize a second subpopulation of biological objects different from the first subpopulation. Then, the operator can return to visualizing the first subpopulation.

In one embodiment, the selection graph is presented in the form of a dendrogram where each node represents a class, the nodes being aligned in strata, each representing the level of membership of the classes represented by said nodes of the stratum, and the branches representing hierarchical links between classes of different levels.

In an alternative embodiment, the selection graph is presented in the form of a sunburst chart comprising concentric rings, each representing a level, a ring having a diameter that is larger the lower the hierarchical level it represents, the classes of a level being fractions of the corresponding ring. The specific shape of the sunburst chart selection graph is particularly advantageous for representing and navigating between the different classes and levels present in the hierarchical structure.

In one embodiment, the length of a said fraction is proportional to the number of points in the corresponding class.

In one embodiment, the fractions representing classes with a direct hierarchical link (i.e., classes belonging to the same extended basin of attraction) are arranged in the same angular sector of the sunburst chart.

In one embodiment, the graphical representation is a scatter plot graph comprising visualization means configured to distinguish the points belonging to two or more selected classes.

In one embodiment, said at least one selection graph is a dynamic graphical representation of the classes and their arrangements in the hierarchical structure, said dynamic graphical representation being configured to display classes of lower hierarchy when the grouping class is selected.

A method according to the invention further preferably comprises one or more of the following embodiments:
p and/or q are greater than 20, preferably greater than 30, preferably greater than 40, and/or less than 80, preferably less than 70, preferably less than 60, preferably equal to 50;
at step a), said distance is representative of a mathematical distance, notably the average distance, between the first point and the neighboring points, said mathematical distance being preferably chosen from: Euclidean distance, Minkowski distance, Manhattan distance, or Mahalanobis distance;
at the end of step a), said densities are subjected to an increasing defined transformation, such as a logarithmic transformation.

In one embodiment, the method further includes receiving a selection of one or more classes from the operator using the selection graph.

In another embodiment, the method further includes providing a medical or research report as output, and/or contacting a person associated with the cytometric events, especially the person from whom the biological fluid on which the cytometric measurements were performed originated, for example, by sending a letter or email, and/or manufacturing a device based on said analysis.

The present invention further relates to a data processing device comprising means for implementing the method according to any one of the embodiments described above.

In more detail, the data processing device is configured for the analysis of a dataset associated with a plurality of biological objects chosen from cells, cellular origin vesicles, acellular microorganisms, and/or biofunctionalized materials; said dataset comprising N cytometric events, each associated with a biological object, each cytometric event being defined by at least two cytometric parameters measured for the corresponding biological object so that the dataset is represented by a cloud (X) of N points in a D-dimensional space; said device comprising:
at least one input configured to receive the dataset associated with a plurality of biological objects;
at least one processor configured to:
   determine, for each point (xᵢ) of said cloud (X), a density (w(xᵢ)) inversely proportional to the sum of distances, raised to a power D, between said point (xᵢ) and a set of neighboring points (Vₚ(xᵢ)) consisting of the p points of said cloud (X) closest to said considered point (xᵢ),
   segment said point cloud (X) into modal segments (Mⱼ), each modal segment (Mⱼ) comprising a modal point (mⱼ), locally presenting a maximum density, and the points of the cloud belonging to the attraction basin of said modal point (mⱼ); the points of the attraction basin being recursively identified based on their density (w(xᵢ)) with respect to the densities of their q nearest neighbors;
   for each modal segment (Mⱼ), determine a persistence (β), said persistence (β) being:
      of infinite value if no adjacent modal segment (Mₜ) to the modal segment (Mⱼ) has a point with a density greater than the density of its modal point (mⱼ); two or more modal segments being adjacent if they share the same density saddle greater than zero; otherwise
      representative of the depth of a density saddle between the modal segment (Mⱼ) and said at least one adjacent modal segment (Mₜ);
   starting from a modal segment (Ma):
      a) determine which, among the considered one (Mⱼ) and its adjacent modal segment(s) (Mₜ), corresponds to the highest attraction basin so as to identify a hierarchical link between the considered modal segment (Mⱼ) and its adjacent modal segment(s) (Mₜ);
      b) fuse the considered modal segment (Mⱼ) and the modal segment(s) with which the considered modal segment (Mⱼ) has a persistence less than or equal to a predefined persistence threshold, so as to define a parent modal segment having a higher attraction basin and having as modal point (mⱼ) the point with the maximum density among the points of the fused modal segments;
      c) iteratively repeat operations a) and b) starting at each new iteration from the parent modal segment;
      so as to determine a hierarchical structure defined based on persistence, said hierarchical structure comprising several levels (Lₖ), each level (Lₖ) defining a segmentation of the cloud (X) into a plurality of classes (Cₖⱼ), each class comprising all the points of one or more modal segments (Mⱼ) and being representative of a group of the plurality of biological objects;
at least one output configured to provide at least the hierarchical structure representative of the different classes of biological objects and their mutual relations.

The present invention further relates to a computer program product comprising instructions which, when the program is executed by a computer, cause it to implement the method according to any one of the embodiments described above.

The present invention further relates to a computer-readable recording medium comprising instructions which, when executed by a computer, cause it to implement the method according to any one of the embodiments described above.

### Definitions

In the present invention, the following terms are defined as follows.

The term “processor” should not be interpreted as being limited to hardware capable of executing software, and generally refers to a processing device, which may, for example, include a computer, a microprocessor, an integrated circuit, or a programmable logic device (PLD). The processor may also encompass one or more graphics processors (GPUs), whether utilized for graphics and image processing or other functions. Additionally, instructions and/or data for executing the associated and/or resulting functionalities may be stored on any processor-readable medium such as, for example, an integrated circuit, a hard drive, a CD (Compact Disc), an optical disc such as a DVD (Digital Versatile Disc), RAM (Random-Access Memory), or ROM (Read-Only Memory). The instructions may be stored in hardware, software, firmware, or any combination thereof.

“Cytometry” generally refers to the detection and/or measurement of cellular characteristics, the basic structural, functional, and biological unit of living organisms. By extension, cytometry also refers to the detection and/or measurement of characteristics of cellular origin vesicles (such as extracellular or intracellular vesicles), or nano- or micrometer-sized particles such as acellular microorganisms (e.g., viruses), or bio-functionalized materials (e.g., microspheres coated with biological molecules such as proteins or nucleic acids, or microspheres coated with bioactive molecules such as an immunomodulatory molecule or a drug).

“Cells” may come from a unicellular or multicellular organism, of prokaryotic or eukaryotic origin, of animal, plant, fungal, protist, bacterial, or archaeobacterial origin. They may be living, dead, or fixed. Cells may, for example, come from solid or liquid tissues (such as bone marrow, blood or lymph, etc.), or body fluids (such as cerebrospinal fluid, urine, bronchoalveolar fluid, etc.).

During the process of acquiring cytometric data, cells, vesicles, or particles may be in suspension (aqueous or non-aqueous, biological or synthetic), or immobilized on a solid support (such as a vial, a cell culture dish with one or more wells, a plate or microplate, a glass slide, a chip, etc.). They may have undergone prior treatment with one or more biological or chemical agents.

The cells, vesicles, or particles to be analyzed may be present within one or more populations.

A “population” is a set of cells, vesicles, or particles exhibiting identical or similar characteristics. A population can be subdivided into different subpopulations exhibiting different secondary characteristics relative to each other.

“Cellular origin vesicles” are vesicles composed of a membrane of cellular origin. These may be intracellular or extracellular vesicles. Extracellular vesicles are typically secreted or excreted by a cell. “Cellular origin vesicles” include, for example, exosomes, ectosomes, microvesicles, microparticles, prostasomes, oncosomes, matrix/calcification vesicles, apoptotic bodies, and other subsets of cellular vesicles.

“Acellular microorganisms” refer to microscopic organisms whose structure is not cellular. “Acellular microorganisms” include, for example, viruses, viroids, and prions.

“Bio-functionalized materials” or “functionalized biomaterials” or “bio-functionalized devices” or “bio-functionalized systems” refer to any type of object comprising a material of synthetic or biological origin coated on its surface, coupled, or bound to one or more molecule(s) or functional group(s) of biological origin.

Non-limiting examples include synthetic or biological origin material such as a nanoparticle (such as a nanobead, nanosphere, or nanocapsule), a microparticle (such as a microbead, microsphere, or microcapsule), a lipid vesicle such as a unilamellar vesicle, a multilamellar vesicle, a lipoplex, a polyplex, a lipopolyplex, a liposome, a niosome, a cochleate, a virosome, an immunostimulant complex (ISCOM®), etc.

Nanoparticles and microparticles, such as beads, spheres, or capsules, can be organic, inorganic, magnetic, or radioactive. For example, nanoparticles or microparticles may be made of metal, silica, alumina, titanium, glass, ceramic, polystyrene, polymethyl methacrylate, melamine, polylactide, latex, dextran, oxide, graphene, magnetic material, radioactive material, or a combination thereof.

Non-limiting examples, to be bio-functionalized, the material may be conjugated with, or coated with, one or more peptide(s), protein(s), antibody (ies), antibody fragment(s), receptor(s), cytokine(s), chemokine(s), toxin(s), oligonucleotide(s), colored or fluorescent molecule(s), amine, carboxyl, or hydroxyl group(s), bioactive molecule(s) (such as an immunomodulatory molecule, a small chemical molecule, a peptidomimetic, a drug), biotin, avidin or streptavidin molecule(s), or a combination thereof.

In the context of the present invention, the term “cytometric”, when qualifying data, parameters, measurements, or events, may refer to cells, cellular origin vesicles, or particles such as acellular microorganisms or bio-functionalized materials.

### BRIEF DESCRIPTION OF THE FIGURES

FIG. 1 is a block diagram illustrating an example of an analysis method according to the invention.

FIG. 2 and FIG. 3 illustrate examples of point density representations obtained according to the method of FIG. 1.

FIG. 4 is an example of a selection graph according to a first embodiment.

FIG. 5 and FIG. 6 illustrate a variant of a selection graph according to a second embodiment.

FIG. 7 illustrates presentation graphs according to one embodiment.

FIG. 8 depicts an example display comprising the selection graph and presentation graphs according to the invention.

FIG. 9 represents an example of using the selection graph to modify the display on the presentation graphs according to the invention.

FIG. 10 is a functional diagram schematically representing a particular mode of a device for analyzing a set of data associated with a plurality of biological objects configured to execute the successive steps of the method of FIG. 1.

FIG. 11 schematically represents a device incorporating the functions of the analysis device of FIG. 10.

### ILLUSTRATIVE EMBODIMENTS

The present description illustrates the principles of the present disclosure. It will therefore be appreciated that those skilled in the art will be able to conceive various arrangements which, although not explicitly described or shown here, embody the principles of the disclosure and are included within its scope.

All examples and conditional language cited herein are intended for educational purposes to assist the reader in understanding the principles of the disclosure and the concepts provided by the inventor to advance the state of the art, and should be interpreted as not limited to those specifically cited examples and conditions.

Furthermore, all statements of principles, aspects, and embodiments of the disclosure, as well as their specific examples, are intended to encompass their structural and functional equivalents. Moreover, it is anticipated that these equivalents include both currently known equivalents and equivalents developed in the future, meaning all elements developed that perform the same function, regardless of their structure.

Thus, for example, those skilled in the art will understand that the schematic diagrams presented herein may represent conceptual views of illustrative circuits implementing the principles of the disclosure. Similarly, it will be appreciated that all flowcharts, flow diagrams, and the like represent various processes that may be essentially represented on a computer-readable medium and thus executed by a computer or processor, whether or not explicitly represented.

The functions of the various elements illustrated in the figures may be provided by the use of dedicated hardware as well as hardware capable of executing software in conjunction with appropriate software. When provided by a processor, the functions may be provided by a single dedicated processor, a single shared processor, or a plurality of individual processors, some of which may be shared.

It is understood that the elements illustrated in the figures may be implemented in various forms of hardware, software, or combinations thereof. Preferably, these elements are implemented in a combination of hardware and software on one or more suitably programmed versatile devices, which may include a processor, memory, and input/output interfaces.

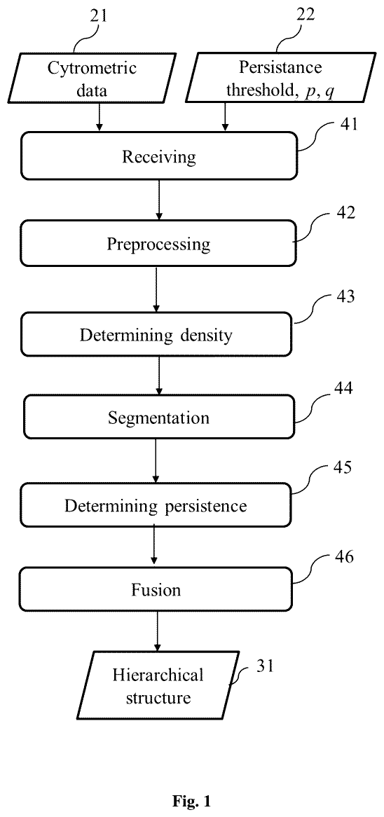

FIG. 1 illustrates an example of implementing a computer analysis method according to the invention.

The method is configured to perform the analysis of a set of data associated with a plurality of biological objects 21 (or cytometric data 21) representable as a cloud X of N points xᵢ in a D-dimensional space, each point xᵢ representing a cytometric event and each dimension representing a cytometric parameter.

The method is configured to output a hierarchical structure 31 representative of the different classes of biological objects, represented in the input dataset, and their mutual relationships.

A hierarchical structure 31 is a multi-level organization Lx, where each level Lₖ defines a respective presentation of the point cloud and/or the data associated with the points of the cloud, i.e., the positions of the points in space and the densities of the points. The higher a level is in the structure, the fewer the number of classes at that level. The structure is hierarchical in that the points of a class Cₖⱼ at a given level Lₖ:
are also comprised in one and only one class C₍ₖ₊₁₎ⱼ at the immediately higher hierarchical level Lₖ₊₁ (if such a level exists above the considered level Lₖ), where the considered class Cₖⱼ is thus the daughter of said class C₍ₖ₊₁₎ⱼ at the level immediately above, itself being the mother of the considered class, and
are also comprised in one or more classes C₍ₖ₋₁₎ⱼ at the immediately lower level L₍ₖ₋₁₎ (if such a level exists below the considered level Lₖ).

The process may include a reception step 41 of cytometric data sets 21 associated with a plurality of biological objects.

Biological objects may include:
cells of animal, plant, fungal, protist, bacterial, or archaeobacterial origin,
cellular origin vesicles chosen from exosomes, ectosomes, microvesicles, microparticles, prostasomes, oncosomes, matrix/calcification vesicles, or apoptotic bodies,
acellular microorganisms chosen from viruses, viroids, and prions, and/or
biofunctionalized materials comprising a synthetic or biological material chosen from a nanoparticle (such as a nanobead, nanosphere, or nanocapsule), a microparticle (such as a microbead, microsphere, or microcapsule), a lipid vesicle (such as a unilamellar vesicle, a multilamellar vesicle, a lipoplex, a polyplex, a lipopolyplex, a liposome, a niosome, a cochleate, a virosome, an immunostimulant complex (ISCOM®)), said synthetic or biological material being coupled to, or coated with, one or more peptide(s), protein(s), antibody fragment(s), receptor(s), cytokine(s), chemokine(s), toxin(s), oligonucleotide(s), colored or fluorescent molecule(s), amine, carboxyl, or hydroxyl group(s), bioactive molecule(s) (such as an immunomodulatory molecule, a small chemical molecule, a peptido-mimetic, a drug), molecule(s) of biotin, avidin, or streptavidin, or a combination thereof.

Cytometric data 21 can be obtained by any method capable of analyzing, determining, or measuring characteristics of a cell, vesicle, or particle. The analysis can focus on a sample comprising a plurality of cells, vesicles, and/or particles. For example, cytometric data 21 can be obtained from a biological sample from an individual, such as one or more organ(s), tissue(s), cell(s), or cell fragment(s) from the individual.

In some cases, the process of acquiring cytometric data 21 may be preceded by a sample preparation process, including, for example, a step of isolating cells, vesicles, and/or particles, or prior isolation of components of said cells.

The sample preparation process comprising the biological objects associated with the cytometric data 21 may include, by way of non-limiting examples, one or more step(s) of magnetic-activated cell sorting (MACS), laser capture microdissection (LCM), manual cell harvesting or micromanipulation, microfluidics, limiting dilution, separation by optical tweezers, electrophoresis, bead separation, immunoprecipitation, immunopanning, labeling, immunostaining, immunofluorescence, multiplexing, and/or the use of biochips.

Cytometric data 21 may be omics or meta-omics technology data, such as genomic, epigenomic, transcriptomic, proteomic, metabolomic, lipidomic, etc. Thus, cytometric data 21 may, for example, relate to the detection and/or quantification of DNA (e.g., genes), RNA, proteins (e.g., cytokines, receptors . . . ), sugars, amino acids, and/or fatty acids present in or on the surface of the cell, vesicle, or particle, or any other molecule contained in the cell, vesicle, or particle. Cytometric data 21 may also relate to the detection of the presence or absence of a particular molecule or assembly in or on the surface of the cell, vesicle, or particle, or to the detection of interactions between molecules present in or on the surface of the cell, vesicle, or particle, or to the detection of interactions between cells and/or objects such as vesicles, microorganisms, or biofunctionalized materials.

As non-limiting and purely illustrative examples, when cytometric data 21 is proteomic in nature, the measured or analyzed cytometric parameters may be the expression level of one or more intracellular or extracellular proteins, or the expression level of one or more receptors or markers on the surface of the cell, vesicle, or particle.

Other examples of cytometric parameters include the size of cells, vesicles, or particles, their density, granularity, morphology/shape, refractive index, membrane composition, molecular content (such as the presence or absence of an intracellular or surface molecule) or their content (such as intracellular content or content of ions, DNA, RNA, . . . ), as well as the redox state of the cell, its status in the cell cycle, its apoptotic state, or its phosphorylation level . . . .

Cytometric data 21 can be obtained through single-cell analysis technologies.

As non-limiting examples, cytometric data 21 can be obtained through flow cytometry (or FACS for “fluorescence activated cell sorting”), PCR-activated cell sorting (PACS), microsphere affinity proteomics (MAP), mass spectrometry, chromatography, CYTOF, spectral cytometry, mass cytometry, imaging cytometry, gene expression profiling on chips (microarray), sequencing, for example DNA sequencing (DNA-seq or single-cell (sc) DNA-seq) or RNA sequencing (RNA-seq or single-cell (sc) RNA-seq), in situ hybridization, and/or microscopy. The term “microscopy” includes atomic force microscopy (AFM), electrochemical detection (EC), scanning electron microscopy (SEM), transmission electron microscopy (TEM), surface plasmon resonance imaging (SPRi), Raman microspectroscopy . . .

Cytometric data 21 can also be obtained by combining several of these techniques. As a purely illustrative example, multiplexed profiling of RNA and protein expression at the single-cell level can be obtained simultaneously by combining the PLAYR technique (proximity ligation assay for RNA), which measures the expression level of a large number of RNAs by flow or mass cytometry, with a technique for detecting surface or internally marked proteins with antibodies.

Another purely illustrative example of a technique for obtaining cytometric data 21 is profiling individual cells isolated in liquid droplets, enveloped by a thin semi-permeable membrane (microcapsules), by high-throughput RNA cytometry, for example by multiplexed RT-PCR.

Cytometric data 21 comprise a plurality of vectors, each comprising the D cytometric parameters measured for a cytometric event associated with a biological object. Each vector can be interpreted as the coordinates of a point in a D-dimensional space. Thus, cytometric data 21 can be represented by a cloud X of N points in a D-dimensional space.

Cytometric parameters subject to cytometric measurements preferably include one or a combination of the following cytometric parameters: size of the particles in the biological fluid on which the cytometric measurements were made, structure of said particles, function of said particles, and any measured parameter reflecting directly or indirectly a characteristic and/or function of a marker of interest (qualitative and quantitative), where the marker of interest may include proteins, nucleic acids, and molecules, alone or in complexes, as well as their interactions.

The number of dimensions D, i.e., the number of cytometric parameters associated with each cytometric event, may be greater than 1, preferably greater than 10, preferably greater than 20, preferably greater than 30, even preferably greater than 40, and/or less than 200.

The number N of points, i.e., the number of biological objects included in the biological fluid subjected to cytometric measurements, is preferably greater than 10,000 and/or less than 109.

The method may involve a preprocessing step of cytometric data 21 (step 42), particularly to facilitate subsequent analysis. Preprocessing may include compensating cytometric data 21, transforming cytometric data 21 into scales suitable for visualization and/or analysis, and/or normalizing cytometric data 21.

For example, compensation can be achieved by multiplying each vector representing a point in the cloud by the inverse of a compensation matrix of size D×D. The compensation matrix can be obtained by any method known to those skilled in the art. Thus, a set of vectors representing the points of the cloud, compensated, can be obtained.

Data transformation may correspond to the application of a mathematical function to the initial or compensated vectors associated with the points of the cloud, said mathematical function may include a linear, logarithmic, bi-exponential function, or hyperbolic arcsine “arcsinh” function. In the illustrated embodiment, the “arcsinh” function is applied to the compensated point cloud.

Finally, data normalization can be performed in a known manner, notably it can be of “Min-max” type.

At the end of step 42, the compensated, transformed, and/or normalized point cloud X is obtained (i.e., the vectors associated with the points of the cloud are compensated, transformed, and/or normalized).

The method of the invention aims to obtain segmentation of the point cloud based on topological persistence, which studies the evolution of the topology of the multilevel set of the density function of the point cloud.

The method includes a step 43 of determining the local density associated with each point in the cloud. In this step, the determination is made, for each point xᵢ of said cloud X, of a density w(xᵢ) inversely proportional to the sum of the distances raised to the power D between said considered point xᵢ and a set Vₚ(xᵢ) consisting of the p points in said cloud nearest to said considered point.

In the present disclosure, a point is considered to be nearby or neighboring the considered point if it is one of the p points in the cloud closest to the considered point. The proximity between two points is defined by a distance between these two points in the D-dimensional space of the cloud. A point may or may not be a neighbor of the considered point depending on the choice of the parameter p and the chosen definition of distance. The proximity between two points can be calculated using Euclidean distance, Minkowski distance, or Manhattan distance. The scope of the invention is not exceeded if other distances are used.

For density calculation, the denominator preferably corresponds to the sum of Euclidean distances (D=2) between the considered point xᵢ and a set Vₚ(xᵢ) consisting of the p closest points of said cloud to the considered point. The distance is preferably an average Euclidean distance between the considered point and the p neighboring points. However, other types of distances can be used, such as Minkowski distance, Manhattan distance, or similar distances.

In the illustrated example, p is set to 50, but other values of p can be chosen, especially depending on the size of the point cloud, the nature of the cytometric data 21, or the intended application of the method.

In the preferred illustrated example, for a point xᵢ in the cloud X, the density w(xᵢ) is calculated up to a constant factor according to the following formula:

$$w ( {x}_{i} ) ∝ \frac{1}{{Σ}_{j = 1}^{p} {‖ {x}_{i} - {V}_{( j )} ( {x}_{i} ) ‖}_{2}^{D}}$$

where ∥.∥₂ is the Euclidean norm on R^{D} and V₍ⱼ₎(xᵢ) represents the j-th nearest neighbor of xᵢ among the p closest neighboring points of the cloud X according to said Euclidean norm.

At the end of step 43, the densities w(xᵢ) of the points in the cloud are preferably subjected to a logarithmic transformation, which advantageously attenuates relative density differences.

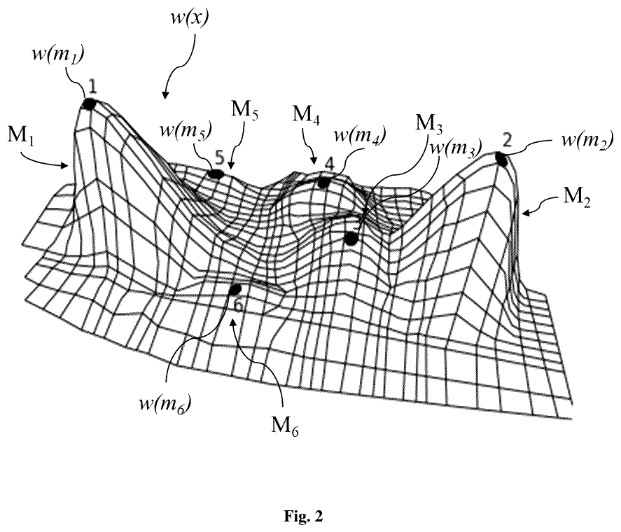

An example of the representation of point density obtained in step 43 is illustrated in FIG. 2. In the illustrated example, the space is in two dimensions, the points of the cloud are in a plane, and the values of the density of said points are represented on a third dimension orthogonal to the plane.

The method then includes a step of segmenting the cloud X into modal segments Mⱼ in order to define groupings (more commonly referred to as “clusters”) of points xᵢ. A modal segment Mⱼ delimits a region of space comprising a group of points from the cloud. This set of points includes a modal point, having a density greater than that of the other points in the modal segment, and other points which, in space, are neighbors of the modal point and have a density lower than that of the modal point. The modal point mⱼ is thus associated with a density, called modal density, which is a local maximum of density, meaning it is higher than that associated with the other points of the modal segment Mⱼ.

The modal segments Mⱼ can be naturally identified with the basins of attraction of the peaks of an approximation of the density function. Intuitively, considering the density function as a landscape, a modal segment Mⱼ is the set of all points xᵢ that flow towards the same local maximum (or peak) along the flow defined by the vector field of the gradient of the density function. Identifying density attraction basins allows for segmenting the point cloud into one or more modal segments Mⱼ, each modal segment Mⱼ comprising a modal point mⱼ associated with a local maximum of the density w(xᵢ), and all points in the cloud belonging to its basin of attraction. The points in the basin of attraction are thus recursively identified based on their density w(xᵢ) in relation to the densities of their q nearest neighboring points.

An adjacent modal segment (or connected) to another modal segment is a modal segment with the same density saddle, said density saddle being greater than zero. Consequently, conversely, two segments having at their common boundary null density values, as no points are found in this region of space, are not considered as adjacent modal segments, in the context of the disclosed method. Locally, on each side of the boundary separating two adjacent modal segments, the points of each of these modal segments are associated with increasing density as one moves away from said boundary. In other words, the boundary defines the bottom of a density saddle.

In other words, two segments are adjacent if there exists a point in the cloud containing at least one point belonging to both modal segments in its neighborhood. The density saddle (or the boundary) is then the point of lowest density among the points containing neighbors within the two adjacent modal segments.

In one embodiment, the following operations can be performed: for each point xᵢ in said cloud, proceeding in decreasing density order, i.e., from the point with the highest density to the point with the lowest density, if the density of said considered point is higher than each of the densities of its q nearest neighbor points V_{q}(xᵢ), a modal segment containing said considered point xᵢ is formed; otherwise, said considered point xᵢ is included in the modal segment containing the point with the highest density among its q nearest neighbor points V_{q}(xᵢ). This operation is based on the knowledge of the distances between pairs of points in the cloud and the density value corresponding to each point in the cloud. Advantageously, the disclosed method only requires knowledge of the (approximate) distances between pairs of cytometric data points 21, as well as approximate estimates of the density at these points. Thus, it is virtually applicable in any arbitrary metric space chosen to represent the cloud of points. Furthermore, the complexity of the method remains reasonable: although the size of the input distance matrix may be quadratic in relation to the number N of points in the cloud, this method only uses a linear amount of main memory in N.

The parameters p and/or q can be greater than 20, preferably greater than 30, preferably greater than 40, and/or less than 80, preferably less than 70, preferably less than 60, preferably equal to 50. The parameters p and q can be identical or different. In the illustrated example, the number q is equal to 50.

In the example illustrated in FIG. 2, six modal segments M1-6 have been identified. As shown, each modal segment can be likened to a mountain, with its peak being a local maximum of density.

The method then includes a step 45 of estimating the persistence for each modal segment Mⱼ.

The persistence β of the modal segment Mⱼ is defined such that:
it has an infinite value if there does not exist at least one adjacent modal segment Mₜ having a point associated with a density greater than the density of its modal point mⱼ or,
it represents the depth of a density col between the modal segment Mⱼ and said at least one adjacent modal segment Mₜ otherwise.

The persistence β of the modal segment Mⱼ having at least one adjacent modal segment can be calculated as the difference between the density of the modal point mⱼ (i.e., the higher density value among the points belonging to the modal segment Mⱼ) and the highest density value among the density values associated with the points of the cloud located in the density col between the modal segment Mⱼ and said at least one adjacent modal segment Mₜ.

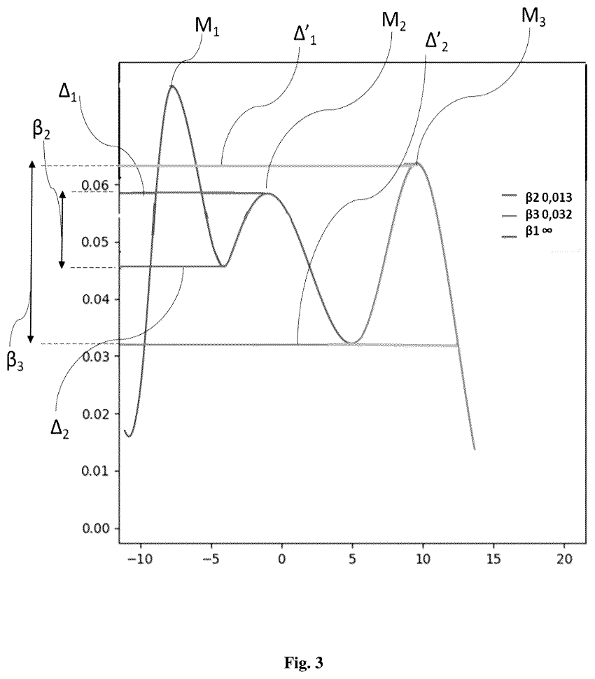

An example of persistence determination is illustrated in FIG. 3, for a simplified representation in which the points of the cloud are associated with a single cytometric parameter, and the density w (x) corresponds to the values on the ordinate axis. In the illustrated example, the persistence β₁ of the modal segment Mⱼ, i.e., the modal segment presenting the local maximum density with the maximum value, is infinite.

The modal segment M₃ is adjacent only to modal segment M₂. Therefore, the persistence β₃ of modal segment M₃ corresponds to the difference between its modal density, i.e., the value of the local maximum of density w(m₃) of the attraction basin of modal segment M₃, and the value of the local minimum density between modal segment M₃ and M₂. This persistence β₃ corresponds to the distance along the ordinate axis separating the two lines Δ′₁ et Δ′₂, in FIG. 3, and is equal, in the example considered, to approximately 0.032.

In the example illustrated in FIG. 3, modal segment M₂ has two adjacent modal segments M₁ and M₃ with density values higher than that of modal segment M₂. The persistence β₂ of modal segment M₂ corresponds to the difference between its modal density, w(m₂) and the highest density value among the two local density minima between the adjacent modal segments, i.e., the difference between the peak of modal segment M₂ and the highest col between modal segment M₂ and M₁. In the illustrated case, persistence β₂ corresponds to the distance along the ordinate axis separating the two lines Δ₁ and Δ₂, in FIG. 3, and is equal, in the example considered, to approximately 0.013.

The next step, Step 46 of the process, is configured to group the modal segments Mⱼ, as well as the points included in the modal segments, based on persistence values calculated in the previous step, in order to construct a hierarchical structure.

The hierarchical structure 31 defined by this Step 46 comprises 1 levels Lₖ, where k ranges from 2 to l. The number l of levels is preferably greater than 2, preferably greater than 3, preferably greater than 4, and/or less than 100, preferably less than 50, even better less than 30.

Each level Lₖ defines a segmentation of the cloud X into H classes Cₖₕ. Each class Cₖₕ groups the points from one or several modal segments Mⱼ defined in Step 44. All modal segments Mⱼ are associated with the classes of a level, with no modal segment belonging to more than 1 class (i.e., no modal segment is shared among multiple classes).

The lower the level in the hierarchical structure 31, the greater the number of classes at that level. The base level L₁ results from grouping all adjacent modal segments that can be grouped into a single common basin of attraction (i.e., coarser segmentation). The final level Lₗ represents the finest segmentation.

To define the classes Cₖₕ at level Lₖ, the modal segments Mⱼ are grouped based on the difference between their respective persistences β and a predefined persistence threshold βₖ^{threshold} (22), which can be global (i.e., having the same value for all levels) or defined differently for each level Lₖ.

Step 46 is configured to arbitrarily choose a starting modal segment Mⱼ that has one or more adjacent modal segments Mₜ. In an alternative embodiment, the starting modal segment Mⱼ is chosen based on the density value of its modal point mⱼ. The starting modal segment Mⱼ can be the modal segment with the modal point m, having the highest density value. According to the method of the invention, a modal segment Mⱼ has multiple adjacent modal segments (Mₜ₁, Mₜ₂, etc.) if the maximum depth of the density col between modal segment Mⱼ and the two or more adjacent modal segments (Mₜ₁, Mₜ₂, etc.) is equal.

For the said starting modal segment Mⱼ, Step 42 is configured to first determine which, among the modal segment Mⱼ and its adjacent modal segment(s) Mₜ, has the highest basin of attraction (i.e., has the modal point mⱼ with the highest density value). The modal segment with a lower basin of attraction is considered to be within the modal segment with a higher basin of attraction, providing information to identify the hierarchical link between the considered modal segments (Mⱼ, Mₜ). The term “height” of the basin of attraction refers to the density value of the modal point of the modal segment (i.e., local maximum of density) associated with said basin of attraction.

Next, the persistence of the modal segment Mⱼ is compared with the predefined persistence threshold value βₖ^{threshold} (i.e., for the first iteration βₗ^{threshold}) for the corresponding level Lₖ. If the persistence of Mⱼ is less than or equal to a predefined persistence threshold βₖ^{threshold} then the modal segment Mⱼ is fused with its adjacent modal segments(s) Mₜ. This fusion defines a new parent modal segment with a higher basin of attraction and with the modal point being the one presenting the maximum density among the points of the fused modal segments. In other words, the points of the cloud X that are attracted by the modal point mⱼ are those belonging to ascending regions (i.e., decreasing density values) that are eventually fused by persistence in the modal segment Mⱼ before being fused into the segment (i.e., basin of attraction) of any other peak with a persistence lower than the predefined persistence threshold βₖ^{threshold}.

In an example, the persistence threshold 22 can be chosen such that each modal segment Mⱼ obtained at step 44, and therefore all its points are associated with a class Cₖₗ of the level Lₗ in the hierarchical structure. The level Lₗ refers to the level with the lowest hierarchy in the hierarchical structure (i.e., the level with the highest granularity). The parent modal segment obtained is associated with a class Cₖ₍ₗ₋₁₎ belonging to the hierarchical level Lₗ directly above. The class Cₖ₍ₗ₋₁₎ thus has a direct hierarchical link with the classes Cₖₗ corresponding to the starting segment M; and its adjacent modal segment(s) Mₜ that have been fused. In particular, the class Cₖ₍ₗ₋₁₎ can be considered as the mother class of these two or more classes of lower hierarchical levels Lₗ.

Finally, these determination and fusion steps are iteratively repeated using the newly defined parent modal segment as the starting modal segment. The same type of hierarchical relationship described above is defined for the parent modal segments created in the subsequent iterations. Consequently, the fusions are performed in order of increasing persistence.

Iterating through these steps allows for the determination of the hierarchical structure 31 based on persistence, thereby providing a representation of groups of biological objects contained within the cytometric data 21.

The number of classes in a level Lₖ depends on the threshold persistence βₖ^{threshold} associated with that level. A preliminary parameterization step is used to determine the threshold persistences βₖ^{threshold} based on the desired number of classes for each level. This preliminary parameterization step involves executing the steps of the method according to the invention for a single threshold persistence value (e.g., a threshold value equal to zero), which allows for the identification of all modal segments present in the point cloud (i.e., no fusion is performed). The values of the threshold persistences βₖ^{threshold} for each level Lₖ are thus chosen based on the analysis of these modal segments and the type of biological objects under examination. These threshold persistence values βₖ^{threshold} are therefore predetermined and independent of the operator, allowing for reproducibility of the process results across different operators. The choice of persistence threshold values thus allows for adapting the granularity of the classes, i.e., the scale of observation of the data according to the type of biological objects under examination, to meet the operator's needs during the display step.

In one embodiment, the step of outputting at least the hierarchical structure 31 further comprises its at least partial representation through an interface accessible to the operator. Preferably, the hierarchical structure 31 is displayed on a screen. Before being presented, it can be stored in computer memory.

The purpose of presenting the hierarchical structure 31 is to allow the operator to choose the classes of interest, grouping exclusively the points that the operator wishes to analyze.

The display step includes the simultaneous display of at least one selection graph 20 and at least one presentation graph 40.

The at least one selection graph 20 is a graphical representation of the classes and their respective relationships in the hierarchical structure 31, and it is adapted to allow the operator to select at least one class Cₖₕ by designating the corresponding class on said graphical representation.

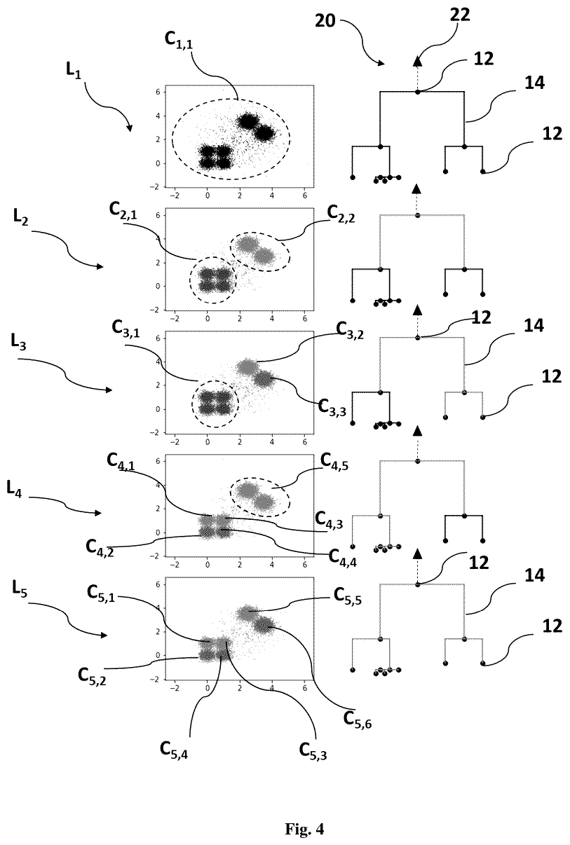

In the same display, the method further represents at least one bi-dimensional or tri-dimensional presentation graph 40 in which each axis is associated with a respective cytometric parameter. In the field of cytometric data analysis 21, it is particularly advantageous for the operator to simultaneously display multiple presentation graphs with different axis pairs. The operator can choose the axes of the presentation graphs 40 via the graphical interface. The presentation graphs 40 are configured to display each of the points in the X cloud belonging to the selected class(es) Cₖₕ using a selection graph 20. An example of simultaneous display is illustrated in FIG. 4, where the left column includes presentation graphs 40 and the right column includes corresponding selection graphs 20.

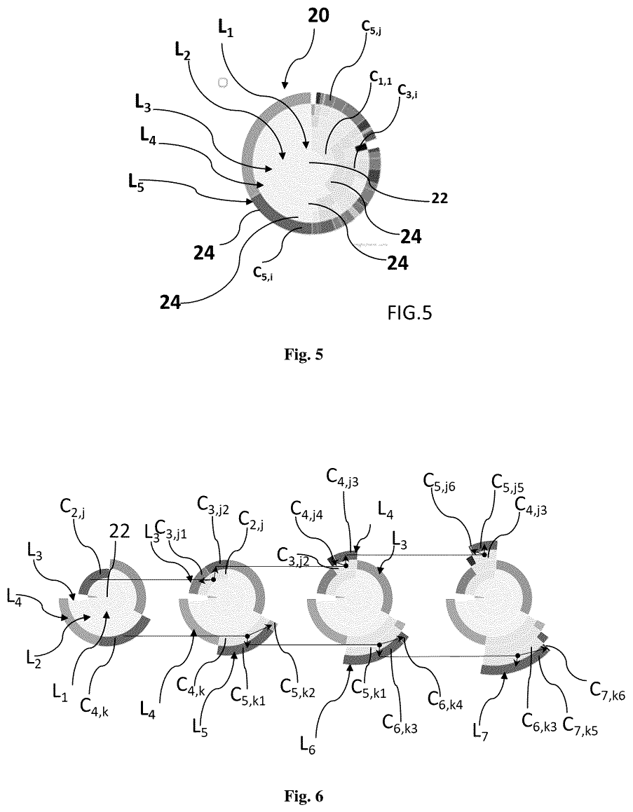

Preferably, the selection graph 20 visually represents the classes by grouping them according to levels. For example, a class may be visually farther from a reference point 22 on the selection graph if it belongs to a level further away from the base level. The reference point could be, for instance, the apex of a pyramid, with each level corresponding to a layer of the pyramid, as shown in FIG. 4, or the center of a circle, with each level corresponding to a ring centered on this nucleus, as depicted in FIG. 5.

In practice, the operator, via the graphical interface, can use a dynamic cursor (i.e., a mouse) to click and thus select one of the classes represented on the graphical representation of the selection graph 20.

In one embodiment, the graphical representation of the selection graph 20 is presented in the form of a dendrogram, which is a diagram representing a tree commonly used to illustrate the arrangement of groups generated by hierarchical clustering. In this dendrogram, each node 12 represents a class Cₖₕ and the branches 14 represent hierarchical links between the classes Cₖₕ. The nodes 12 belonging to the same level Lₖ can be aligned in strata.

In an example illustrated in FIG. 4, the selection graph 20 takes the form of a dendrogram, with levels L₁₋₅ representing different branching levels. Each node 12 represents a class Cₖₕ and each branch 14 represents a hierarchical link between a mother class Cₖₕ and a daughter class C₍ₖ₊₁₎ₕ.

In one embodiment, the selection graph 20 is presented in the form of a sunburst chart. This chart is depicted as a disk divided into a plurality of sectors or “quadrants”, with each sector representing all, and only the points (and/or associated data) of a respective class from the highest, base level. Preferably, the angle of a sector is proportional to the number of points in the class from the base level it represents. The sunburst chart comprises concentric rings, with each ring representing a level of the hierarchical structure 31. Particularly, there is a core associated with level L₁ and l−1 concentric rings 24, each representing other levels Lₖ for k ranging from 2 to l−1 having a hierarchy lower than level L₁. In this sunburst chart, a ring has a diameter that increases with representing a lower hierarchical level.

The classes Cₖₕ of the same level Lₖ can be represented as fractions of the corresponding ring, and each fraction extends into the sector containing classes from a higher hierarchical level before a direct or indirect hierarchical link. Two classes have a direct hierarchical link if only one branch connects them, and they have an indirect hierarchical link if they are connected by at least two branches.

Preferably, a fraction representing a class extends around the center of the disk in an arc whose opening angle is proportional to the number of points in the class represented by the fraction.

FIG. 5 illustrates an example of a sunburst chart, where the levels are concentric rings 24 and are closer to their center as they represent aggregated classes, with the classes Cₖₕ of a level Lx being fractions of the corresponding ring 24 for that level Lₖ.

In this preferred embodiment, the diameter of a ring 24 is larger as it represents a level L₂₋₅ further away from the base level L₁.

Preferably, for all levels, the fractions of a ring from a lower hierarchical level (i.e., daughter) representing daughter classes C₍ₖ₊₁₎ₕ of a mother class Cₖₕ are depicted within the same angular sector as the fraction representing the said mother class Cₖₕ in the ring associated with the higher hierarchical level (i.e., mother). This way, the fractions of daughter classes C₍ₖ₊₁₎ₕ overlap externally the fraction associated with the mother class Cₖₕ.

As an illustrative and non-limiting example, when the analyzed objects are cells, a mother class may correspond to a cellular population of lymphocytes. This lymphocyte population (mother class) can be subdivided into different cellular subpopulations (corresponding to daughter classes of the mother class), such as for example a T lymphocyte subpopulation and a B lymphocyte subpopulation, these two types of subpopulations presenting different secondary cytometric parameters. On the sunburst selection graph 20, the different cellular subpopulations are represented by a “daughter” ring surrounding the “mother” ring.

Furthermore, these T and B lymphocyte subpopulations can in turn be subdivided into further subpopulations. For example, within the T lymphocyte subpopulation, analysis of cytometric parameters may distinguish a CD4+ T lymphocyte subpopulation (T lymphocyte expressing the CD4 protein on its surface) and a CD8+ T lymphocyte subpopulation (T lymphocyte expressing the CD8 protein on its surface). The selection graph 20 can present the entire hierarchical structure 31 or, preferably, only a portion of the hierarchical structure 31.

The selection graph 20 may further include a visualization tool adapted to highlight the selected class(es) on the graphical representation. The visualization tool may, for example, be configured to modify the color, shape, or size of the class representation on the selection graph 20 (i.e., node 12 or fraction).

In one embodiment, the selection graph 20 is further configured to provide a dynamic graphical representation of the classes and their arrangements within the hierarchical structure. This dynamic graphical representation can be configured to display lower hierarchy classes when the class that groups them is selected on the selection graph 20.

In this embodiment, the selection graph 20 defaults to displaying the classes of the base level. This way, when the operator clicks on the representation of a “mother” class from the base level, the interface presents a representation of the daughter classes of that mother class. More generally, when the operator clicks on the representation of a “mother” class Cₖₕ from any level Lx displayed on the screen, the computer presents the daughter classes C₍ₖ₊₁₎ₕ of that mother class Cₖₕ.

Thanks to this embodiment, the operator can “expand” any class Cₖₕ that is presented by the selection graph 20 and that does not belong to the last level of the hierarchy. By “expanding” a class, we mean presenting the daughter classes of that class. The operator can thus focus on cytometric events that interest them (see FIG. 6), which is particularly advantageous for analysis.

Preferably, the selection graph 20 only presents the classes of the base level, and the classes developed from the classes of said base level.

Conversely, thanks to this embodiment, the operator can collapse all daughter classes of a mother class that are presented by the selection graph. By collapsing a set of daughter classes, we mean erasing the representations of said daughter classes from the screen. The operator can thus return to a more macroscopic representation of the cytometric events of the daughter classes (see FIG. 6).

FIG. 6 illustrates, from left to right, an example of class expansion, and from right to left, an example of class collapsing according to the invention. In this example, selecting classes C_{2,j} and C_{4,k} from levels L₂ and L₄, respectively, allows access to their respective daughter classes, namely C_{3,j1}, C_{3,j2}, C_{5,k1} and C_{5,k2} from levels L₃ and L₅.

Similarly, expanding classes C_{3,j2} and C_{5,k1} displays their daughter classes C_{4,j3}, C_{4,j4}, C_{6,k3} and C_{6,k4}.

Similarly, by selecting classes C_{4,j3} and C_{6,k3}, this allows the operator to access their respective daughter classes C_{5,j5}, C_{4,j6}, C_{7,k5} and C_{6,k6}.

Advantageously, the operator can easily choose the classes of interest across different levels of the hierarchical structure.

A expandable class-by-class selection graph 20, rather than level by level, advantageously identifies a portion of the hierarchical structure consisting of only the classes of interest chosen from different levels, or a sub-hierarchical structure of interest, without needing to display the same granularity on other classes that do not contain cytometric events relevant to the use case, as illustrated in FIG. 6.

The graphical representation of the classes and their respective relationships in the hierarchical structure 31 of the selection graph 20 is thus used to select the points of only the classes that the operator wishes to visualize in the presentation graph(s) 40. When a class is selected on the selection graph 20, all its points are represented on each of the presentation graphs 40.

The presentation graphs 40 can be represented in the form of scatter plots. A scatter plot in two or three dimensions is simply a projection of the selected points of the cloud onto a plane or a 3D space associated with two or three chosen cytological parameters respectively for the plane or the 3D space.

The number of presentation graphs is preferably greater than 1, 2, 3, and/or less than 200, 100, 50, 30, or 10.

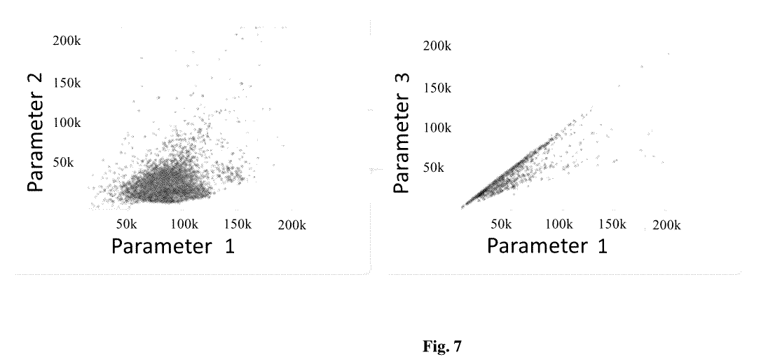

The points of the same class can all be represented in the same way. These presentation graphs 40 therefore preferably include visualization means configured to distinguish the points belonging to two or more selected classes (FIG. 7). These visualization means allow the points of the same class to be presented identically on the different presentation graphs and specific to said class, allowing the operator to identify them directly. For example, the points of the same class have a shape, dimension, and/or color specific to the class.

When the points of the cloud are represented differently depending on their class membership, for example, having a specific color for the class, the representation associated with a level shows the classes as groups of points of different colors. The granularity is coarser the higher the level represented is in the hierarchical structure 31. Conversely, the classes belonging to levels located at the bottom of the hierarchical structure 31 provide a representation with finer granularity of the populations and subpopulations present in the cytometric data 21. Selecting a level thus allows the operator to choose the scale at which he wants to visualize the cytometric events.

In the example illustrated in FIG. 7, the points of the same class have the same color specific to that class.

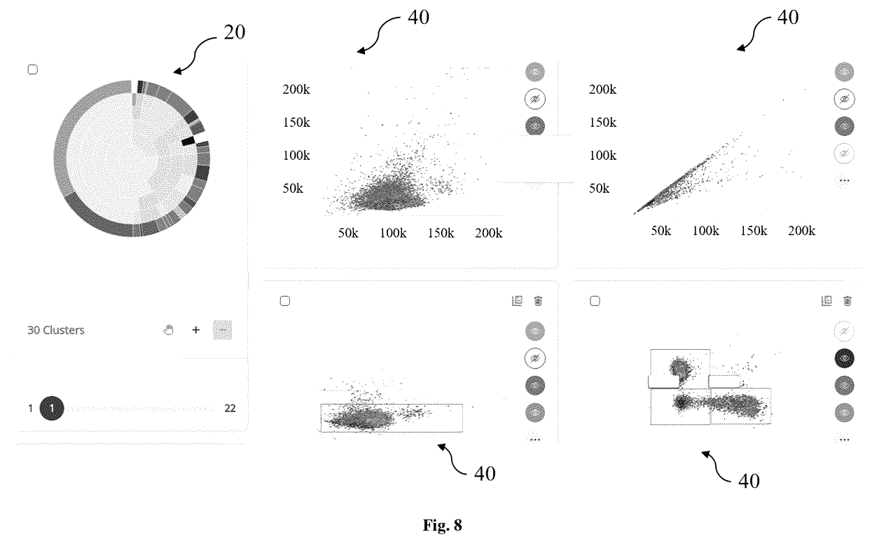

FIG. 8 shows an example of display according to the invention in which the selection graph 20 is a sunburst graph and four representation graphs 40 of the scatter plot type are shown on the same screen of the operator interface.

If the operator wants to zoom in on a mother class to reveal its daughter classes, the points must be represented with an appearance specific to the daughter class to which they belong. The visualization means are therefore configured to modify the appearance of the points of the mother class so that each group of points belonging to one of its daughter classes has the same characteristic appearance of said class (i.e., differs from or other daughter classes) when the operator selects the daughter classes on the selection graph 20.

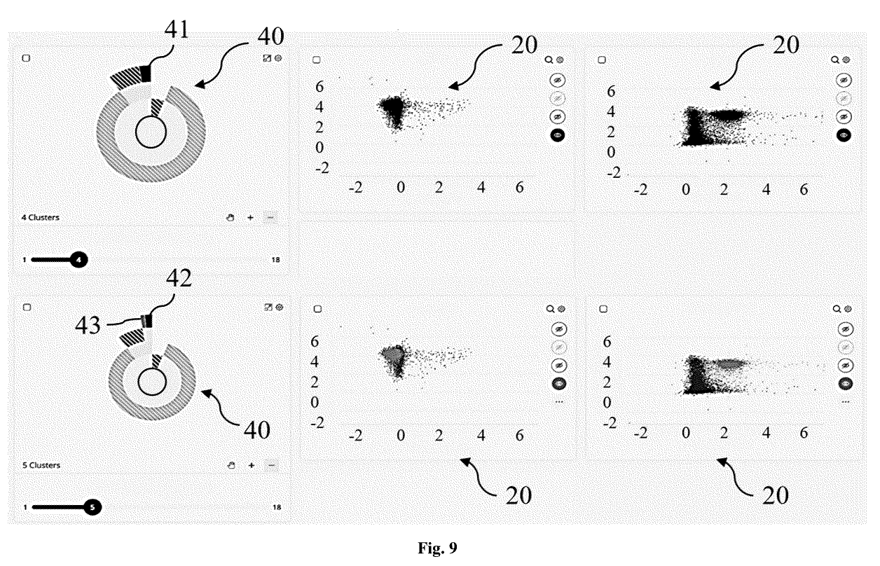

FIG. 9 shows an example of modification of the display following the interaction of the operator with the selection graph 40. The left column of this figure shows a first configuration of the display in which the class associated with the fraction of the ring 41 of the selection graph has been selected by the operator and the two presentation graphs 20 therefore show the points associated with the biological objects included in the selected class. In this example, the points are displayed in the same color as the fraction of the ring 41. The right column shows how the display has been modified by the operator who chose to “zoom in” on the mother class associated with the fraction of the ring 41 to visualize the two daughter classes, represented by the two fractions of the ring 42 and 43. This action of zooming in by selecting the two fractions of the ring 42 and 43, also allows to modify the display of the points associated with the two daughter classes simultaneously or quasi-simultaneously with the selection of the daughter classes. The display of the points on the presentation graphs 20 is modified so as to be able to distinguish between the points belonging to the two classes. In this example, the color of the points has been changed (black and gray on the presentation graph 20 in the right column). Thanks to this display, the operator obtains information at the same time on the community relations between selected subtypes of biological objects (i.e., hierarchical link between the mother class and the daughter classes) and specific information regarding their cytometric parameters.

Advantageously, the method of the invention allows the operator to select, in at least one presentation graph, the class(es) of interest that he wishes to visualize or not visualize the points thanks to the interaction with the selection graph. Advantageously, it is thus notably possible not to display points considered as irrelevant, for example noise made up of cell debris, matrix, or crystals for example. In conclusion, the presentation mode implemented by the method of the present invention helps the operator to perform the technical task of evaluating the state of the system from which the cytometric data 21 originate (i.e., patient's health status) through a human-machine interaction process.

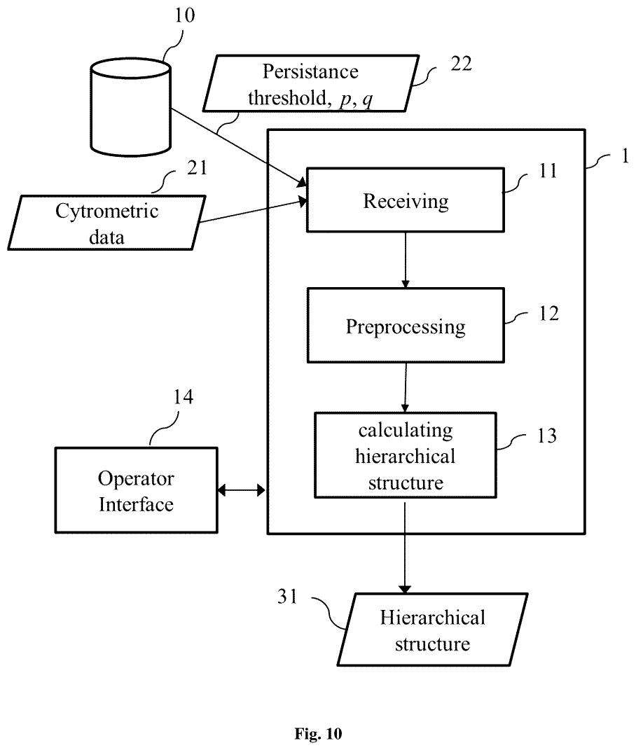

The present invention relates, among other things, to a data processing device 1 configured to implement the steps of the method described above (FIG. 10).

Although the device I currently described is versatile and equipped with several functions that can be executed alternatively or in any cumulative manner, other implementations within the scope of the present disclosure include devices having only parts of the functionalities described herein.

The device 1 is advantageously an apparatus, or a physical part of an apparatus, designed, configured, and/or adapted to execute the mentioned functions and produce the effects or results mentioned. In alternative implementations, the device 1 is realized in the form of a set of apparatuses or physical parts of apparatuses, whether they are grouped in the same machine or in different machines, possibly remote. The device 1 may, for example, have functions distributed over a cloud infrastructure and be available to users as a cloud-based service, or have remote functions accessible via an API.

In the following disclosures, modules should be understood as functional entities rather than physically distinct components. They can therefore be materialized either as grouped together in the same tangible and concrete component, or distributed across several of these components. Likewise, each of these modules may itself be shared among at least two physical components. Furthermore, the modules are implemented in the form of hardware, software, firmware, or any mixed form thereof.

The device 1 includes a module 11 for receiving cytometric data 21 as well as predefined hyperparameters for the method 22 (i.e., p, q, and/or predefined persistence threshold(s)) stored in one or more local or remote databases 10. The latter can take the form of storage resources available from any type of suitable storage means, which may include, in particular, RAM or EEPROM (Electrically-Erasable Programmable Read-Only Memory) such as Flash memory, possibly within an SSD (Solid-State Disk). Alternatively, the hyperparameters 22 are received via a communication network.

The device 1 optionally includes a module 12 for preprocessing cytometric data 21 configured to perform one or more of the operations associated with step 42 of the method.

Furthermore, the device 1 includes a module 13 for calculating the hierarchical structure 31 configured to perform one or more of the operations associated with steps 43, 44, 45, and 46 of the method.

The device 1 interacts with an operator interface 14, through which information can be entered and retrieved by a user. The operator interface 14 includes any suitable means for entering or retrieving data, information, or instructions, including visual, tactile, and/or auditory capabilities that may include one or more of the following well-known means to those skilled in the art: a screen, a keyboard, a trackball, a touchpad, a touchscreen, a speaker, a voice recognition system.

The device 1 may also include a display module 15 configured to perform the display step using the operator interface 14.

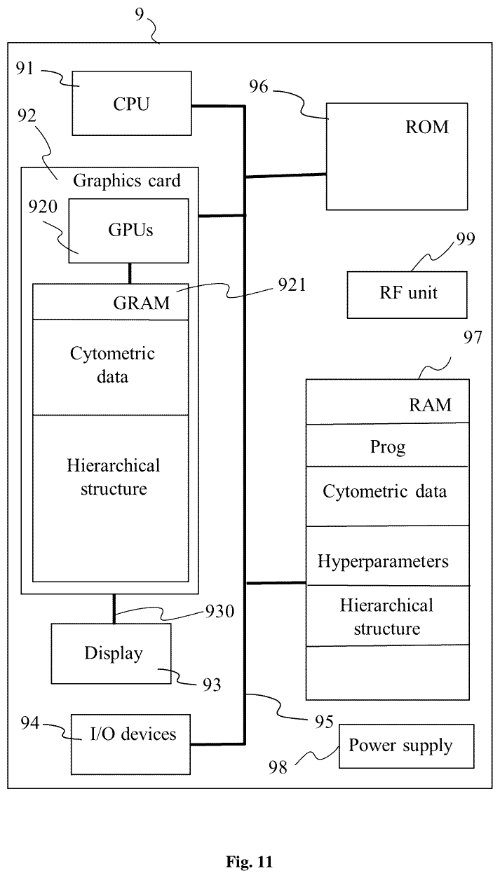

A particular device 9, visible in FIG. 11, implements the device 1 described above. It may correspond, for example, to a workstation, a laptop computer, a tablet, a smartphone, or a head-mounted display (HMD).

This device 9 is suitable for analyzing a dataset associated with a plurality of biological objects. It includes the following elements, interconnected by an address and data bus 95 that also carries a clock signal:
a microprocessor 91 (or CPU);
a graphics card 92 comprising multiple graphics processing units (or GPUs) 920 and a graphics random-access memory (GRAM) 921;
a ROM type non-volatile memory 96;
a random-access memory (RAM) 97;
one or more input/output (I/O) devices 94 such as a keyboard, a mouse, a trackball, a webcam; other methods of inputting commands such as voice recognition are also possible;
a power source 98; and
a radiofrequency unit 99.

In a variant, the power supply 98 is external to the device 9.

The device 9 also includes a display device 93 of the direct display screen type directly connected to the graphics card 92 to display synthesized images calculated and composed in the graphics card. The use of a dedicated bus to connect the display device 93 to the graphics card 92 offers the advantage of having much higher data transmission rates and therefore reducing latency for displaying images composed by the graphics card.

In a variant, a display device is external to the device 9 and is connected to it by a cable or wirelessly to transmit display signals. The device 9, for example through the graphics card 92, includes a transmission or connection interface adapted to transmit a display signal to an external display means such as, for example, an LCD or plasma screen or a projector. In this regard, the radiofrequency unit 99 can be used for wireless transmissions.

It should be noted that the term “register” used below in the description of memories 97 and 921 may designate in each of the mentioned memories, a memory area of low capacity (a few binary data) as well as a memory area of large capacity (allowing to store a whole program or to calculate or display all or part of the data representative of the data). Similarly, the registers represented for RAM 97 and GRAM 921 can be arranged and constituted in any manner, and each of them does not necessarily correspond to adjacent memory locations and can be distributed differently (which covers in particular the case where a register includes several smaller registers).

At power-up, the microprocessor 91 loads and executes the instructions of the program contained in the RAM 97.

As will be understood by those skilled in the art, the presence of the graphics card 92 is not mandatory and can be replaced by complete processing by the central unit and/or simpler visualization implementations.

Furthermore, the device 1 can be implemented differently from standalone software, and a device or set of devices comprising only parts of device 9 can be operated through an API call or a cloud interface.

### EXAMPLES

Many algorithms have been tested in Weber, L. M. and Robinson, M. D. (2016), Comparison of clustering methods for high-dimensional single-cell flow and mass cytometry data. Cytometry 2016, 89 (12): 1084-1096 (doi: 10.1002/cyto.a.23030).

However, a method according to the invention provides a better compromise between reliability and acceptability than these algorithms.

In particular, the inventors compared, for different datasets (Levine32, Levine13, Samusik01, SamusikAll), the reliability obtained with different algorithms tested by Weber and that obtained with a method according to the invention.

Reliability was measured by the F-score, which is a classic measure of agreement between the results of an algorithm and the ‘ground truth’.

The following Table 1 summarizes the obtained F-scores.

| Dataset | Best previous score | Method according to the invention |
|---|---|---|
| Levine32 | 0.780 (FlowSOM) | 0.738 |
| Levine13 | 0.518 (FlowMeans) | 0.495 |
| Samusik01 | 0.707 (FlowSOM) | 0.613 |
| SamusikAll | 0.702 (FlowSOM) | 0.671 |

This table shows that a method according to the invention provides reliability comparable to that of the best algorithms from the prior art.

However, it not only provides reliable segmentation but also results that are acceptable to any cytometric analysis specialist, notably because:
these results are particularly robust, meaning they are reproducible;
these results do not stem from the implementation of an artificial intelligence-based algorithm, whose conclusions are difficult to explain, but are based on densities, which allows for an objective explanation of the cytometric event groupings in the classes;
these results do not require subjective initial parameterization of the process;
these results can be organized in a manner well adapted to cytometry, namely in a hierarchical form, whereas cells themselves are traditionally classified hierarchically.

A method according to the invention is thus considered to present the best compromise between reliability and acceptability (resulting notably from excellent interpretability) for a cytometric analysis specialist.

As is evident, the invention provides a solution for analyzing cytometric data that:
provides relevant segmentation,
is easily interpretable, verifiable, and acceptable to any cytometric analysis specialist;
allows for very flexible navigation among cytometric events;
enables rapid selection of representations of cytometric events.

The analysis of biological fluid thus offers an improved compromise between reliability, efficiency, and acceptability.

1-14. (canceled)

15. A computer-implemented method for analyzing a set of data associated with a plurality of biological objects chosen from cells, cellular origin vesicles, acellular microorganisms, and/or biofunctionalized materials; said set of data comprising N cytometric events, each associated with a biological object, each cytometric event being defined by at least two cytometric parameters measured for the corresponding biological object so that the set of data is represented by a cloud of N points in a D-dimensional space, the method comprising:
determining, for each point of said cloud, a density inversely proportional to the sum of distances, raised to a power D, between said point and a set of neighboring points consisting of p points of said cloud closest to said considered point,
segmenting said cloud of points into modal segments, each modal segment comprising a modal point, locally presenting a maximum density, and the points of the cloud belonging to the attraction basin of said modal point; the points of the attraction basin being recursively identified based on their density vis-à-vis the densities of their q nearest neighboring points;
for each modal segment, determining a persistence, said persistence being:
of infinite value if no adjacent modal segment to the modal segment has a point with a density greater than the density of its modal point; two or more modal segments being adjacent if they share a same density saddle greater than zero; otherwise
representative of the depth of a density saddle between the modal segment and said at least one adjacent modal segments;
starting from a modal segment:
a) determining which, among the considered one and its adjacent modal segment(s), corresponds to the highest attraction basin so as to identify a hierarchical link between the considered modal segment and its adjacent modal segment(s);
b) fusing the considered modal segment and the modal segment(s) with which the considered modal segment has a persistence lower than or equal to a predefined persistence threshold, so as to define a parent modal segment having a higher attraction basin and having as modal point the point presenting the maximum density among the points of the fused modal segments; and
c) iteratively repeating operations a) and b) starting at each new iteration from the parent modal segment;
so as to determine a hierarchical structure defined based on persistence, said hierarchical structure comprising multiple levels, each level defining a segmentation of the cloud into a plurality of classes, each class comprising all the points of one or more modal segments and being representative of a group of the plurality of biological objects;
outputting at least the hierarchical structure representative of the different classes of biological objects and their mutual relationships.

16. The method according to claim 15, wherein the segmentation of the point cloud into modal segments comprises, for each point, proceeding from the point with the highest density to the point with the lowest density:
comparing the density of said point with that of its q nearest neighboring points,
if the density of the corresponding point is greater than the density of all its q nearest neighboring points, defining a modal segment comprising the considered point, otherwise
including said considered point in a modal segment comprising the point with the highest density among its q nearest neighboring points.

17. The method according to claim 15, wherein, in the presence of one or more adjacent modal segments to the modal segment, determining the persistence as a difference between the density of the modal point and the highest density among the densities of the points in the cloud in the density col between the modal segment and said at least one adjacent modal segment.

18. The method according to claim 15, wherein the step of providing as output at least the hierarchical structure further comprises displaying:
at least one selection graph, said selection graph being a graphical representation of the classes and their respective relationships in the hierarchical structure, said selection graph being adapted to select at least one class by designating the corresponding class on said graphical representation;
at least one bi-dimensional or tri-dimensional presentation graph, each axis being associated with a respective cytometric parameter, said at least one presentation graph being configured to display the points of the cloud belonging to said at least one selected class using the selection graph.

19. The method according to claim 18, wherein the graphical representation is presented in the form of:
a dendrogram where each node represents a class, the nodes being aligned in strata each representing the level of membership of the classes represented by said nodes of the stratum, and where the branches represent hierarchical links between classes of different levels; or
a sunburst chart comprising concentric rings each representing a level (Lₖ), with a ring having a diameter that is larger the lower the hierarchical level it represents, and the classes of a level being fractions of the corresponding ring at that level.

20. The method according to claim 18, wherein the selection graph further comprises a visualization mean configured to highlight said at least one selected class on the graphical representation.

21. The method according to claim 18, wherein the graphical representation is a scatter plot graph comprising visualization means configured to distinguish the points belonging to two or more selected classes.

22. The method according to claim 18, wherein said at least one selection graph is a dynamic graphical representation of the classes and their arrangements in the hierarchical structure, said dynamic graphical representation being configured to display classes of lower hierarchy when the class that groups them is selected.

23. The method according to claim 15, wherein the biological objects are:
animal, plant, fungal, protist, bacterial, or archaebacterial cells,
cellular origin vesicles selected from exosomes, ectosomes, microvesicles, microparticles, prostasomes, oncosomes, matrix/calcification vesicles, or apoptotic bodies,
acellular microorganisms selected from viruses, viroids, and prions, and/or
biofunctionalized materials comprising a synthetic or biological material selected from a nanoparticle (such as a nanobead, nanosphere, or nanocapsule), a microparticle (such as a microbead, microsphere, or microcapsule), a lipid vesicle (such as a unilamellar vesicle, multilamellar vesicle, lipoplex, polyplex, lipopolyplex, liposome, niosome, cochleate, virosome, immunostimulant complex (ISCOM®)), said synthetic or biological material being coupled to, or coated with, one or more peptide(s), protein(s), antibody (ies), antibody fragment(s), receptor(s), cytokine(s), chemokine(s), toxin(s), oligonucleotide(s), colored or fluorescent molecule(s), amine, carboxyl, or hydroxyl group(s), bioactive molecule(s) (such as an immunomodulatory molecule, a small chemical molecule, a peptidomimetic, a drug), biotin, avidin, or streptavidin molecule(s), or a combination thereof.

24. The method according to claim 15, wherein the cytometric parameters are selected from the size of the biological objects, their density, granularity, morphology, shape, refractive index, membrane composition, molecular content, content of a molecule, and/or the level of expression of a molecule.

25. The method according to claim 24, wherein one of the measured cytometric parameters is the level of expression of one or more protein(s), receptor(s), marker(s), and/or the level of expression of one or more nucleic acid(s) such as DNA or RNA.

26. The method according to claim 15, wherein the cytometric data 21 are obtained by flow cytometry (or FACS for “fluorescence activated cell sorting”), by PCR-activated cell sorting (or PACS), by microsphere affinity proteomics (MAP), by mass spectrometry, by chromatography, by CYTOF, by spectral cytometry, by mass cytometry, by imaging cytometry, by gene expression profiling on chips (microarray), by sequencing (e.g., scDNA-seq or scRNA-seq), by in situ hybridization, and/or by microscopy.

27. A computer program product comprising instructions which, when the program is executed by a computer, cause it to implement the method according to claim 15.

28. A data processing device comprising means for implementing the method according to claim 15.
